(19) United States
(12) Patent Application Publication
(10) Pub. No.: US 2025/0130173 A1
(43) Pub. Date: Apr. 24, 2025

(54) DETECTION USING SERS PROBES

(71) Applicant: United Kingdom Research and Innovation, Swindon Wiltshire (GB)

(72) Inventors: Pavel Matousek, Oxfordshire (GB); Sara Mosca, Oxfordshire (GB); Nicholas Stone, Exeter (GB)

(21) Appl. No.: 18/836,105

(22) PCT Filed: Feb. 7, 2023
(86) PCT No.: PCT/GB2023/050265

§ 371 Date: Aug. 6, 2024

(30) Foreign Application Priority Data
Feb. 8, 2022 (GB) 2201614.1

(51) Int. Cl. G01N 21/65; G01N 33/483

(52) U.S. Cl. CPC G01N 21/658; G01N 33/4833

(57) ABSTRACT

Methods and apparatus for detecting one or more properties of a sub-surface volume of a diffusely scattering sample are disclosed, where the diffusely scattering sample comprises surface enhanced Raman Spectroscopy (SERS) probes, the SERS probes being spectrally responsive to a stimulus. The stimulus is provided in a varying form at the sub-surface volume, and during the varying stimulus, probe light is directed to an entry region on a surface of the sample. A portion of the probe light is collected, including elements of said probe light Raman scattered from said SERS probes, at a collection region on the surface of the sample, the collection region being spaced from the entry region. The collected Raman scattered elements are then measured, and variations in the measured Raman scattered elements which are induced by the variations in the stimulus are detected. One or more properties may then be detected based on the detected variations in the measured Raman scattered elements. For example, the variations in the stimulus may comprise changes in the stimulus over time at the sub-surface volume, and the variations in the measured Raman scattered elements which are induced by the variations in the stimulus may then comprise changes in the measured Raman scattered elements over time.

The present invention relates to methods and apparatus for detecting one or more properties of a sample comprising surface enhanced Raman spectroscopy (SERS) probes, for example when these are located in a sub-surface volume of a diffusely scattering sample such as human or animal tissue. The methods and apparatus may for example be used to detect the presence or concentration of such SERS probes in particular states, such as when Raman reporter molecules forming part of the probes are bound to particular chemical targets.

### INTRODUCTION

Surface enhanced Raman spectroscopy (SERS) provides enhancement, typically by several orders of magnitude, of the Raman scattering cross section from Raman reporter molecules when these are bound to a nanostructured metal surface, such as the surface of a discrete sub-micron metal nanoparticle, or an electrochemically roughened metal layer deposited on a larger surface. Because of the cross-section enhancement, SERS has found many applications, such as the detection of low-abundance biomolecules including antibodies and other proteins in bodily fluids.

However, despite the signal enhancement available using SERS, there remain situations in which obtaining sufficient signal to noise of the Raman signal is challenging.

It would be desirable to address problems and limitations of the related prior art.

### SUMMARY OF THE INVENTION

The inventors have observed that, although SERS provides enhancement of Raman scattering, and the ability to link that enhancement to the detection of Raman reporter molecules coupled to noble metal nanoparticles or other SERS probes, further improvement of the Raman signal may often be necessary or desirable. For example, either relatively low concentrations of such SERS probes, or requirements for faster detection at adequate signal to noise ratios, may make detection difficult. In some applications, the SERS probes may be buried within a significant depth of a diffusely scattering or similar material which can further make detection difficult.

The invention therefore provides an “active SERS” technique in which variations of a Raman signal received from SERS probes (in particular variations in spectral shape, form or intensity are detected through their synchrony with variations of an external perturbation or stimulus used to modify the Raman spectral response of the SERS probes. The technique can be used to enhance considerably the sensitivity of detection of the SERS probes, and by suitable focus of the stimulus can also provide high spatial resolution of detection which may be desirable in imaging applications.

In one aspect, the invention provides a method of detecting one or more properties of a sample which comprises SERS probes which are spectrally responsive to a stimulus, the method comprising: providing the stimulus in a varying form to the SERS probes; during the varying stimulus, directing probe light to the SERS probes and collecting a portion of the probe light, including elements of said probe light Raman scattered from said SERS probes; measuring the collected Raman scattered elements; detecting variations in the measured Raman scattered elements which are induced by variations in the stimulus; and detecting the one or more properties based on the detected variations in the measured Raman scattered elements. Detecting variations in the measured Raman scattered elements which are induced by variations in the stimulus may for example comprise detecting variations in the spectral shape or form of the Raman spectra of the SERS probes that are in synchrony with, or are correlated with, the variations in the stimulus.

In particular, the SERS probes may be spectrally responsive to a stimulus, in that features, shape or form, or intensity, of their Raman spectra change in response to the stimulus, and in particular exhibit variations in response to variations in the stimulus. The invention then provides a method of detecting one or more properties of a sample which comprises such SERS probes, the method comprising: providing the stimulus in a varying form to the SERS probes to modify their spectral response; during the varying stimulus, directing probe light to the SERS probes and collecting a portion of the probe light, including elements of said probe light Raman scattered from said SERS probes; measuring the collected Raman scattered elements for example as Raman spectra; and detecting variations in the measured Raman scattered elements or spectra which are induced by variations in the stimulus. The detected variations in the measured Raman scattered elements may typically be variations, induced by the stimulus, in the shape or form, or intensity, of the Raman spectra of the SERS probes. The method may then also comprise detecting the one or more properties based on the detected variations in the measured Raman scattered elements or spectra.

More particularly, if the SERS probes are comprised or located within a diffusely scattering sample, and more particularly within a sub-surface volume of such a sample, then the method may comprise a method of detecting one or more properties of the sub-surface volume of the diffusely scattering sample, or of detecting the SERS probes themselves, the SERS probes being spectrally responsive to the stimulus. In particular, the method may be a method of detecting one or more properties of a sub-surface volume of a diffusely scattering sample which comprises SERS probes, the SERS probes being spectrally responsive to a stimulus, the method comprising: providing the stimulus in a varying form at the sub-surface volume (for example to modify the spectral response of the SERS probes); during the varying stimulus, directing probe light to an entry region on a surface of the sample, and collecting a portion of the probe light, including elements of said probe light Raman scattered from said SERS probes, at a collection region on the surface of the sample; measuring the collected Raman scattered elements, for example as Raman spectra; and detecting variations in the measured Raman scattered elements which are induced by the variations in the stimulus. The detected variations in the measured Raman scattered elements may typically be variations, induced by the stimulus, in the shape or form of the Raman spectra of the SERS probes. The method may then also comprise detecting the one or more properties based on the detected variations in the measured Raman scattered elements.

The variations in the stimulus mentioned above may comprise changes in the stimulus over time at the sample or at the sub-surface volume, and the variations in the measured Raman scattered elements which are induced by the variations in the stimulus may comprise changes in the measured Raman scattered elements over time. However, the variations could also be implemented in other ways for example using spatial variations.

Note that the varying stimulus is not provided by the probe light giving rise to the Raman spectral features to be detected, although light of a other wavelengths may be used as the stimulus.

The detecting of variations in the measured Raman scattered elements which are induced by variations in the stimulus may comprise detecting such variations that are in synchrony with, or are correlated with, the variations in the stimulus. Such synchrony or correlations may comprise temporal synchrony or correlation, allowing for any lags between the two, or more complex relationships such as a monotonic decrease or increase in the intensity of Raman scattered elements arising from the SERS probes.

The variations in the measured Raman scattered elements or Raman spectra induced by variations in the stimulus may comprise variations in one or more of the intensity, the width, and the wavenumber of one or more Raman spectral features of the measured Raman scattered elements, for example including one or more new Raman bands appearing, or Raman bands disappearing, for example due to molecular conformational changes induced by the stimulus.

The step of detecting the one or more properties may comprise forming a component spectrum representing variations in the measured Raman scattered elements induced by variations in the stimulus, and determining the one or more properties from the component spectrum. Such a component spectrum may for example comprise a spectrum of variation in Raman scattered elements expressed as a function of wavelength or wavenumber.

In some examples, the component spectrum may be a principal component of the measured Raman scattered elements, formed using principal component analysis, or a partial least squares component of the measured Raman scattered elements, formed using partial least squares analysis, the principal or partial least squares component representing changes in the measured Raman scattered elements which are induced by changes in the varying stimulus.

In other examples the component spectrum may represent a correlation between the measured Raman scattered elements and the stimulus. For example, the component spectrum may represent a difference between the measured Raman scattered elements when the stimulus in a first state for example an “on” state, and the measured Raman scattered elements when the stimulus in a second state for example an “off” state. A single cycle or multiple cycles of the two stimulus states could then be used.

The step of detecting one or more properties of the sample may be detecting properties of the SERS probes, or of conditions at the SERS probes, or of properties of the sample influencing or affecting the Raman spectra of the SERS probes. For example, the detected one or more properties of the sample or of the sub-surface volume may comprise one or more of: particular states of the SERS probes; binding of the SERS probes to one or more target species; a temperature at the SERS probes; a pH at the SERS probes; a density or quantity of the SERS probes; and presence or absence of the SERS probes.

The SERS probes may take various forms, but typically each SERS probe may comprise a metal nanoparticle, typically a noble metal nanoparticle or at least a nanoparticle with a noble metal surface. Multiple nanoparticles such as dimers, trimers and so on, or generally clusters, can also or instead be used, thereby benefiting additionally from strong enhancements within one or more SERS ‘hot spots’ present at the proximal points between the nanoparticles. These SERS ‘hot spots’ are also likely to exhibit high sensitivity to external perturbations due to their inherently high nanoparticle distance sensitivity and as such constitute favourable designs for the formation of suitable nanoparticle assemblies likely to exhibit heightened spectral sensitivity (e.g. SERS signal intensity) to the varying stimulus. The nanoparticles can be free floating (mobile) in solution or confined to a substrate. Particular locations on a roughened or patterned metal substrate can also act as suitable SERS probes.

Each metal nanoparticle or other SERS probe form may then carry one or more Raman reporter molecules, such that the elements of the probe light Raman scattered from the SERS probes are at least principally elements of the probe light scattered from the Raman reporter molecules. Detecting the SERS probes in the sub-surface volume may in particular comprise detecting the SERS probes to be in one or more particular states, wherein the particular states optionally comprise one or more states of the Raman reporter molecules or other aspects of the SERS probes being bound, or not being bound, to one or more target species to be detected through detection of the SERS probes.

The varying stimulus may take various forms as long as it causes suitable corresponding detectable variations in the Raman scattered elements. The varying stimulus may for example be or comprise one or more of: a non-optical or non-light stimulus; an ultrasound stimulus; a magnetic stimulus; a time varying stimulus; a time varying ultrasound field; a time varying magnetic field stimulus; a time varying light field stimulus; and a time varying microwave or terahertz wave field stimulus. The time varying stimulus could also be or comprise a time varying temperature at the SERS probes provided by more direct thermal control.

Note that the stimulus is typically separate to, or distinct from, the probe light used to obtain the measured Raman scattered elements, for example being generated by a stimulus source which is separate or distinct from the source of the probe light. For example, whereas the probe light will typically be narrow band laser light for example in the near infrared, the stimulus may take a wide variety of different forms including ultrasound, magnetic fields, heat, electromagnetic radiation of various forms and so forth as discussed in more detail below. To this end, the stimulus may specifically not comprise the probe light, or may specifically not comprise light of the same wavelength as the probe light.

If the time varying stimulus is a time varying ultrasound field, then this may be generated by a high-intensity focussed ultrasound transducer optionally having a central aperture. In this way, optical coupling to the sample for applying the probe light and/or collecting the scattered probe light may be through this aperture. In the case of a diffusely scattering sample, one or both of the entry region and collection region may be located within or visible through the central aperture.

The time varying stimulus may be varied according to various patterns or cycles, for example using a sinusoidal, square or triangular wave of intensity, strength, field direction, and so forth. A repeating cycle time of the time varying stimulus for inducing variations in the detected Raman scattered element of the same or a corresponding cycle time may typically be between about 0.001 and 100 seconds (0.01 to 1000 Hz), or between about 0.1 and 10 seconds (0.1 to 10 Hz), although other cycle times may be used. The number of effective cycles of the time varying stimulus may vary widely for example with tens or hundreds of repeat cycles of the variation pattern, down to as little as a single Off-On, On-Off, or Off-On-Off cycle.

Note that in many forms, a particular stimulus may be driven or may oscillate at other, typically much higher frequencies, which do not correspond to the cycle time or frequency of the stimulus variation and corresponding detected or detectable induced variations in the Raman scattered elements discussed here. For example, if the stimulus is a magnetic field stimulus, then that magnetic field stimulus may comprises an AC magnetic field driven at high frequency for example in the kHz to MHz ranges, while the variations in the stimulus used to induce corresponding variations in the detected Raman scattered elements will typically have cycle times of the order of about 0.1 to 10 or 0.001 to 100 seconds as mentioned above. Similarly, although an ultrasound or electromagnetic radiation field has a very high intrinsic frequency, the time variations of such stimuli referred to in the present context should lead to corresponding detected variations in the Raman scattered elements, so are of rather lower frequency for example falling in the cycle time ranges noted above.

Where the sample is diffusely scattering, the entry and collection regions may be disposed in various ways and using various geometries. In some such geometries the entry and collection regions maybe coincident or overlapping, and in other embodiments they may be spatially separated or offset. For example, in order to optically sample a bulk of the sample between the entry and collection regions, these may be disposed on opposite sides of the sub-surface volume, to implement a forward scattering or transmission geometry.

In other arrangements a SORS type geometry may be used, typically as a backscatter type geometry, for example including separately detecting the SERS probes for each of a plurality of different spatial offsets between the entry and collection regions, and associating the detected SERS probes for each different spatial offset with a different depth or distribution of depth within the sample. The one or more properties may then be detected at each different depth or distribution of depth, based on the detected variations in the measured Raman scattered elements. In such arrangements, the entry and collection regions may typically be spatially offset by an offset in the range from 1 mm to 50 mm, or in the range from 3 mm to 20 mm.

Typically for a diffuse sample, the sub-surface region and the SERS probes to be detected may be located at distances beneath the surface of the sample which are in the ranges of: at least 1 mm; from 1 mm to 80 mm; and from 3 mm to 50 mm. In the case of a diffusely scattering sample, such a sample may typically have a diffuse scattering transport length of less than 4 mm, or of less than 2 mm. The diffusely scattering sample will typically be a solid sample, although it could be a gel or a liquid, and could comprises a variety of biological tissues.

The described techniques may particularly be used to provide much higher spatial resolution of detection of properties of the sample or of the SERS probes themselves than is generally possible in a diffusely scattering sample. For example, if detecting SERS probes at a depth of around ten millimetres, it would usually be difficult to provide a spatial resolution even of comparable size, say ten millimetres. However, because the described techniques rely on an externally applied stimulus to induce detectable variations in the Raman response of the SERS probes, if such a stimulus is limited or focussed to a smaller volume, then detection of the properties of the sample or of the SERS probes themselves can be limited to such a smaller volume. To this end, the stimulus may be provided in a sub-surface volume of the sample such that a volume of the sample, within which the stimulus has a magnitude greater than 1/e of its peak magnitude within that volume, or alternatively a volume of the sample in which the SERS probes can be detected using the implemented technique, may be limited to no more than 1000 mm³ or no more than 100 mm³ or no more than 1 mm³ of the sample.

Especially if a focus or spatial limitation of the stimulus, and/or the optics to deliver and collect the probe light, are used to limit the detection area or volume of the technique, the described methods may be used to detect a one, two or three dimensional scan or map of the detected properties of the sample. The described methods then comprise repeating the detection of the properties for a plurality of different surface regions of or sub-surface volumes within sample, wherein for each such surface region or sub-surface volume the stimulus in time varying form is provided to that region or sub-surface volume for the detection of the sample properties in that region or sub-surface volume.

The described techniques may be applied to various different sample types in which or within which the SERS probes may be present, or are present and may be in a particular state to be detected. For example, the sample may be an in-vivo, and diffusely scattering portion of a human or animal body, and the surface of the sample may then be skin of the in-vivo portion.

The invention also provides apparatus for detecting one or more properties of a sub-surface volume of a diffusely scattering sample which comprises SERS probes, the SERS probes being spectrally responsive to a stimulus as already noted above, the apparatus comprising: a stimulation source arranged to provide the stimulus in a varying form at the sub-surface volume; delivery optics arranged to direct probe light to an entry region on a surface of the sample during the varying stimulus, and collection optics arranged to collect a portion of the probe light, including elements of said probe light Raman scattered from said SERS probes, at a collection region on the surface of the sample; a spectral detector arranged to measure the collected Raman scattered elements; and an analyser arranged to detect variations in the measured Raman scattered elements which are induced by the variations in the stimulus. The detected variations in the Raman scattered elements may typically be variations, induced by the stimulus, in the shape or form of the Raman spectra of the SERS probes. Note also that the entry and collection regions may be spaced from each other in various ways as discussed herein.

The analyser may also be arranged to detect the one or more properties based on the detected variations in the measured Raman scattered elements.

The varying stimulus may be provided by the stimulation source as one or more of: a non-optical or non-light stimulus; an ultrasound stimulus; a magnetic stimulus; a time varying stimulus; a time varying ultrasound field, using an ultrasound transducer; a time varying magnetic field typically using one or more electrical coils (for example wound around an air core or ferromagnetic core); a time varying light field for example provided by a laser, super luminescent diodes, or other light sources; and a time varying microwave of terahertz wave field.

If the stimulation source comprises an ultrasound transducer then this may be a high-intensity focussed ultrasound (HIFU) transducer. Conveniently, such a transducer may have a central aperture, and one or both of the entry region and collection region may then be arranged to direct probe light to/collect light from the sample within or through the central aperture. However, in other embodiments one or both of entry and collection regions could be laterally spaced from the transducer, for example if there is no central aperture.

The apparatus may also comprise one or both of said SERS probes and the sample, for example a diffusely scattering sample within which the SERS probes are located.

The invention also provides computer program code arranged to carry out or implement described aspects of the method and apparatus, and one or more computer readable media carrying such computer program code. For example the computer program code may be arranged to carry out the following steps: receiving a timing signal representing a time varying stimulus which is provided to surface enhanced Raman Spectroscopy (SERS) probes within a sample (for example within a sub-surface volume of the sample, wherein the sample may be a diffusely scattering sample), the SERS probes being spectrally responsive to the stimulus; receiving data representing measured Raman scattered elements of probe light collected from said SERS probes during application of the time varying stimulus; detecting from the timing signal and the data representing measured Raman scattered elements, variations in the measured Raman scattered elements which are induced by the variations in the stimulus; and detecting one or more properties of the sample based on the detected variations in the measured Raman scattered elements.

### BRIEF DESCRIPTION OF THE DRAWINGS

Embodiments of the invention will now be described, by way of example only, with reference to the accompanying drawings, of which:

FIG. 1 illustrates schematically apparatus for detecting one or more properties of a sub-surface volume of a diffusely scattering sample by Raman detection of SERS probes by using a varying stimulus;

FIG. 2 shows how the analyser of FIG. 1 may be implemented using principal component, or partial least squares, or a similar multivariate analysis technique;

FIG. 3 shows how the analyser of FIG. 1 may be implemented using a spectral subtraction technique;

FIG. 4 shows how the analyser of FIG. 1 may be implemented by reducing the Raman spectra to a reduced or single time series spectral parameter;

FIG. 5 shows how an ultrasound transducer may be used to provide the time varying stimulus at the same time as Raman scattering from SERS probes within a diffusely scattering sample;

FIG. 6 shows how electrical coils may be used to provide a magnetic time varying stimulus at the same time as Raman scattering from SERS probes within a diffusely scattering sample;

FIG. 7 illustrates how properties of a sample may be detected using a time varying stimulus and Raman detection where the SERS probes are not buried or contained in a diffusely scattering sample, in this case where the SERS probes are used to detect species in a flow cell;

FIG. 8 illustrates an experiment demonstrating synchrony or correlation between properties of Raman spectral features in light scattered from SERS probes, and intensity of an ultrasound stimulus;

FIG. 9 shows detected Raman spectral features in the form of a Raman spectrum (upper curve) of SERS probes in the experiment of FIG. 8, and a component spectrum in the form of a principal component (lower curve) of the variation of that spectrum over time under variations in the stimulation;

FIG. 10 shows variations in temperature (upper curve), ultrasound status (middle curve) and the score of the principal component of FIG. 6 within the acquired spectra over time, of the experiment of FIGS. 8 and 9; and

FIG. 11 provides a flow diagram of a method according to the invention in accordance with the apparatus and methods illustrated in FIGS. 1 to 10.

### DETAILED DESCRIPTION OF EMBODIMENTS

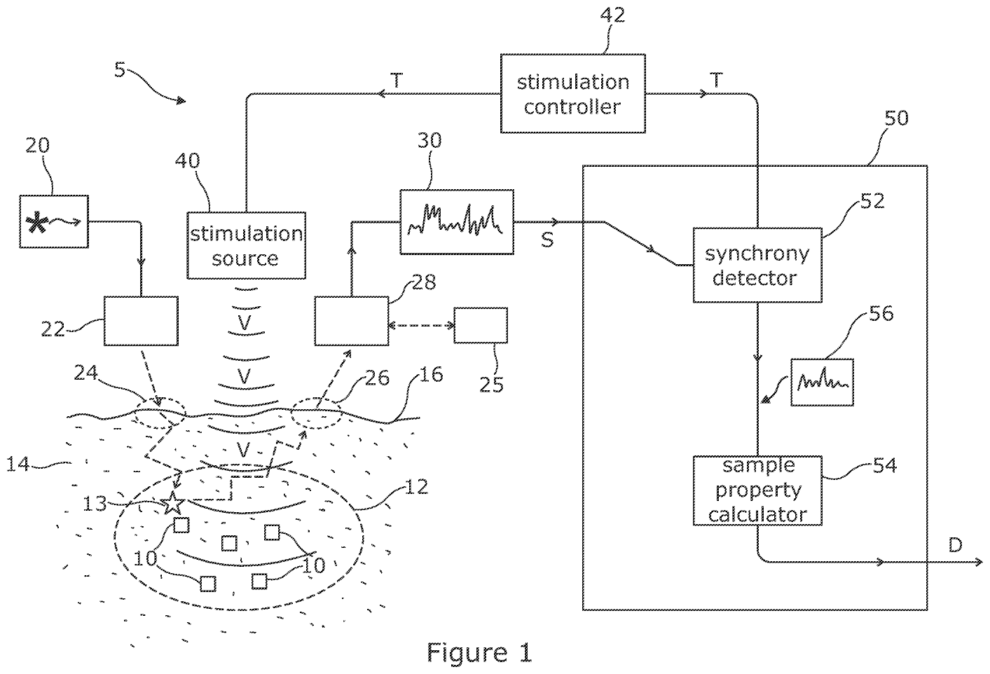

Referring now to FIG. 1 there is shown, schematically, apparatus 5 for detecting properties of a sample, and typically a diffusely scattering sample 14, using surface enhanced Raman spectroscopy (SERS) probes 10, for example in a sub-surface volume 12 of the sample. The sample may for example be an in-vivo portion of a human or animal body, such as an arm, breast or finger, and an exposed surface 16 of the sample 14 may then be skin of that portion, although the invention may equally be used with a variety of other samples and sample types as discussed in more detail below. The diffusely scattering sample may typically be a solid or gel sample, although it may comprise fluid regions such as blood vessels and/or lymph vessels if the sample comprises an in-vivo or ex-vivo portion of a human or animal body.

The SERS probes 10 may typically be provided by metal nanoparticles, such as noble metal nanoparticles or a SERS probe substrate. The SERS probes, and therefore the sub-surface volume, may be located at a variety of distances beneath the surface 16, for example from about 3 mm to 50 mm or from about 1 mm to 80 mm beneath the surface. If the sample is an in-vivo portion of a human or animal body then the SERS probes may have been placed within the sub-surface volume by injection through the skin, by distribution within the blood or lymph, or in various other ways.

The apparatus 5 is arranged to detect elements of probe light which have been Raman scattered from the SERS probes 10. Notably, the SERS probes 10 have properties such that at least some of the Raman scattered elements are spectrally responsive to an imposed stimulus, other than or separate to or distinct from the probe light itself, where the spectral response could involve changes in properties such as intensities or relative intensities compared to other parts of the Raman scattered elements, positions, sizes and/or widths of particular Raman spectral peaks under changes in the imposed stimulus. The stimulus could for example comprise a non-light stimulus such as ultrasound, magnetic fields or microwaves, but could also or instead comprise light, but typically not including light of the same wavelength or waveband as the probe light. The Raman spectral response or Raman spectra of the SERS probes may then change for example as a result of local temperature changes due to such stimuli, or through other mechanisms as discussed in more detail below.

The SERS probes may typically comprise Raman reporter molecules, for example as Raman reporter molecules bound to the surface of noble metal nanoparticles. The apparatus 5 may then particularly be arranged to detect elements of the probe light which have been Raman scattered from the Raman reporter molecules, where the Raman scattering may be dependent on one or more particular target states of the reporter molecules. Such states may be for example states in which the reporter molecules are bound, or are not bound, to a target such as a biomolecule which may be present in the sample, or in which they experience certain conditions for examples levels of pH or temperature.

To this end, the Raman reporter molecules may typically have properties such that the elements of probe light Raman scattered from the molecules changes under certain conditions, such as when a particular biomolecule or other target to be detected within the sample is bound, or is not bound, to the Raman reporter molecule, under changes in pH. However, in some embodiments the SERS probes may function without any of basic Raman reporter molecules, by providing information through their presence, absence or intensity in the detected signals.

The apparatus 5 of FIG. 1 comprises a probe light source 20 arranged to deliver probe light to delivery optics 22 which in turn are arranged to direct the probe light to an entry region 24 on the surface 16 of the sample. The probe light then scatters within the sample 14, including within the sub-surface volume 12, and some of the probe light then emerges through a collection region 26 of the surface 16 to be collected by collection optics 28. The entry and collection regions 24, 26 may be provided according to a variety of different geometries and spatial relationships to each other on the surface 16 of the sample, but may typically be coincident, overlapping, or mutually spaced. Such mutual spacing may be by around 1 to 50 mm with optional variable spacing in order to provide a depth profile of detection of the SERS probes, or located on opposing surfaces of the sample as discussed in more detail below.

A small proportion of the scattering events of the probe light within the sample 14 are Raman scattering events 13 at a SERS probe 10, giving rise to Raman scattered probe light characteristic of the SERS probe 10 or its current state, or more typically of a Raman reporter molecule bound to the SERS probe. The collected probe light is therefore passed to a spectral detector 30 where such Raman scattered elements are detected, typically as a Raman spectrum.

The probe light source 20 may typically comprise one or more lasers, so that the probe light is provided by the probe light source 20 as a laser beam. Suitable wavelengths for the probe light may be in the near infra red or visible red regions, for example with a wavelength of around 500 to 2000 nm. Whether or not the probe light is laser light, the probe light should be sufficiently narrowband in nature for useful Raman spectral features to be detectable in the collected light.

The delivery optics 22 and collection optics 28 may each comprise one or more suitable lenses, mirrors and/or filters. For example, optical filters may be provided in the delivery optics to suppress unwanted laser side bands in the probe light generated by the probe light source 20, and the collection optics will typically comprise one or more filters to suppress the elastically scattered probe light and minimise entry of this and other background signals into the spectral detector 30 where it would otherwise reduce the quality of detection of the Raman scattered elements. In some embodiments, the delivery and collection optics may share or comprise common optical components.

The spectral detector 30 may comprise a spectrometer arranged to detect probe light which has been Raman scattered from the SERS probes, and output corresponding detected Raman spectral features S. The Raman spectral features S may typically be output as an extended Raman spectrum of the collected light, for example extending across some or all of the Raman fingerprint region typically found around 600-1800 cm⁻¹ in wavenumber shift from the wavenumber of the probe light. However, a smaller spectral region may be detected and output as the detected Raman spectral features S, for example if all of the Raman spectral features of interest are found in such a smaller spectral region. In some embodiments, one or more discrete spectral features may be detected and output as the detected Raman spectral features S, instead of an extended spectrum, for example using one or more discrete photodetector elements in combination with suitable optical filters to output properties of one or more particular spectral lines or wavenumbers.

Although the detected Raman spectral features S typically comprise spectral features of interest arising from the SERS probes when present in sufficient concentration, and/or when present in one or more particular states in sufficient concentration, the Raman spectral features Swill typically also comprise other spectral components, including for example background noise, Raman spectral features from the material or matrix of the sample, Raman spectral features from other parts of the system such as a sample holder, optical components, and so forth. It would therefore be desirable to improve the detection of the SERS probes, and therefore detection of properties of the sample, even when their Raman spectral signature is very weak or difficult to detect above background noise and other signals.

To this end, the apparatus 5 also comprises a stimulation source 40 arranged to direct a varying stimulus into the sub-surface volume 12 of the sample 14, typically through the surface sample 16. The stimulation source 40 could for example be an ultrasound transducer arranged to direct ultrasound into the sample 14. A stimulation controller 42 then controls the stimulation source 40 for example using a timing signal T to provide a suitable variation of the stimulus. Typically, the stimulus will be controlled to vary over time, and one such time variation could be a square wave on-off cycle such that the ultrasound or other stimulus is turned on and off, or selectively directed to the sub-surface volume according to such a time variation. However, in some embodiments, other modes of variation of the stimulus could also or instead be used, for example a spatial variation where the stimulus varies in spatial manner and Raman spectral features S are detected from respective different portions of the sub-surface volume of the sample.

A suitable time variation of the stimulus could for example be to turn the ultrasound transducer or other stimulation source on and off with a repetition period of a few seconds. Other timing signals could be sinusoidal, saw-tooth, or triangular in form. Generally speaking, the stimulus could be applied in a repeating pattern having a cycle time which is between about 0.001 and 100 seconds, but the choice of such a cycle time may depend on factors such as how quickly a Raman spectrum of adequate signal to noise can be measured from the collected probe light, and how quickly the sub-surface volume of the sample, or the SERS probes themselves, typically provide a sufficient spectral response to the stimulus. In some embodiments as little as a single cycle of variation of the stimulus may be sufficient, for example as a single Off-On or On-Off cycle of the stimulus.

As discussed above, the SERS probes 10, at least when in a state where they are to be detected, have properties such that at least some of the Raman scattered elements of the probe light are spectrally responsive to the stimulus, where the spectral response could involve changes or variations in properties such as intensities or relative intensities, positions, sizes and/or widths of particular Raman spectral peaks under changes or variations in the imposed stimulus. Such spectral responses are then captured within the Raman spectral features S output by the spectral detector 30, and are used to detect one or more properties of the sub-surface volume of the sample based on the variations in these measured Raman scattered elements have been, or are estimated to have been, induced by the variations in the stimulus.

The variations in the measured Raman spectral feature that have been induced by the variations in the stimulus may be identified as, or estimated to be, those which are determined to be in synchrony with, or correlated with, the variations in the stimulus. This synchrony or correlation may typically be identified as a function of time, with or without a lag or time difference, or with a more complex relationship between the two.

Detecting the SERS probes from synchrony or correlation between the stimulus and the measured Raman spectral features light permits much more sensitive detection of the SERS probes, which is particularly advantageous when the Raman spectral signal of the SERS probes is weak, or buried in significant background and noise, as is likely to be the case when SERS probes 10 in a sub-surface volume of a diffusely scattering sample 14 are to be detected.

In the arrangement of FIG. 1, the detected Raman spectral features S and timing signal Tare passed to an analyser 50 for further processing in order to detect one or more properties of the sub-surface volume of the sample using the SERS probes 10. To this end, the analyser 50 as depicted in FIG. 1 comprises a synchrony detector 52 and a sample property calculator 54. The synchrony detector 52 receives the Raman spectral features S and optionally also the timing signal T, and determines from these one or more synchronous features 56 which represent variations in the Raman spectral features S which are estimated to have been induced by the variations in the stimulus as represented by the timing signal T. The synchronous features 56 may comprise one or more specific spectral features such as a change in intensity at one or more specific wavenumbers, changes in wavenumber of one or more specific spectral peaks or similar, or may comprise wider spectral features such as a derived component spectrum of the detected Raman spectrum which represents changes across the whole of the Raman spectrum estimated to have been induced by variations in the stimulus.

The sample property calculator 54 then uses these synchronous features 56 to determine one or more properties of the sub-surface volume 12 of the sample which are output as results D. Note that the synchronous features 56 may only be detectable when the SERS probes 10 are present in sufficient concentration, or when the SERS probes are present in sufficient concentration in a particular state (for example bound to a particular biomolecule or other target), in the sub-surface volume.

The determined properties may comprise for example, a measure of the presence, absence or concentration of the SERS probes, and measure of the presence, absence or concentration of the SERS probes which are in a particular state for example a state of binding to a particular target molecule, a measure of temperature, a measure of pH, and so forth.

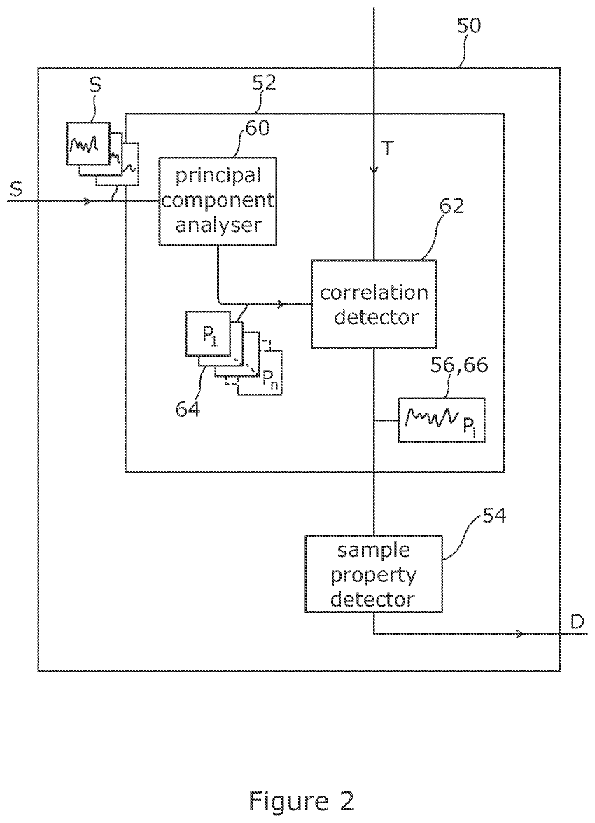

FIG. 2 illustrates in more detail one way in which the synchrony detector 52 may be implemented within the analyser 50. In this arrangement, the synchrony detector comprises a principal component analyser 60 and a correlation detector 62. The principal component analyser 60 accepts as input a time series of Raman spectra S from the spectral detector 30 and determines a hierarchy of principal components P₁ . . . Pₙ which cumulatively represent the time variations in the spectra over some time interval that preferably encompasses multiple cycles of variation of the stimulus. The correlation detector 64 then evaluates the contribution that each of these principal components makes to the spectra of the time series, and evaluates the degree of correlation between that contribution, as a time series, and the timing signal T. The selected principal component P, giving rise to the highest correlation can then be selected as that which best represents variations in the Raman spectra which are induced by variations in the stimulus.

The selected principal component P; is then output by the synchrony detector 52 as synchronous features 56 in the form of a component spectrum 66 of the Raman spectrum S, for input to the sample property calculator 54 which operates to calculate one or more properties of the sub-surface volume of the sample from the component spectrum 66. By way of example, a peak in the component spectrum 66 at a particular wavenumber or wavelength may represent a significant variation in the Raman spectrum S at that wavenumber which has been induced by variation of the stimulus, therefore indicating presence of the SERS probes at sufficiently high concentration or the presence of the SERS probes in a particular bound state at sufficiently high concentration. Similarly, an adjacent peak and trough in the component spectrum 66 may represent a wavenumber shift in a particular peak of the Raman spectrum under variations in the stimulus. Similarly, the magnitude of the component spectrum 66 across the full or part of the wavelength range may represent a particular concentration of the SERS probes or the SERS probes in a particular state.

The component spectrum 66 may also convey information about the environment surrounding the SERS probes through information imprinted on the component spectrum through further spectral alterations (Raman spectral shifts, bandwidth changes, new bands appearing or disappearing, anti-Stokes to Stokes band intensity ratios), for example reflecting the local chemical and physical state of the SERS probe environment (e.g. chemical bonding, polarity, pH, temperature). Other principal components correlating with the stimulus could also be selected for example by the correlation detection so as to provide multiple component spectra 66, and inspected in a similar manner to determine properties of the sample.

Although in FIG. 2 a principal component of the Raman spectral features S received from the spectral detector 30 is selected as a component spectrum 66 or synchronous features 56 representing variations in the spectral features which are induced by the stimulus, other data analysis and multivariate methods may be used instead or in addition to derive similar component spectra or synchronous features. For example, a partial least squares method may be used to determine a hierarchy of partial least squares components, or a classical least squares or a multivariate curve resolution analysis may be used instead to determine a hierarchy of other generated components, one of the components then being selected by the correlation detector 62 for best synchrony or correlation with the timing signal.

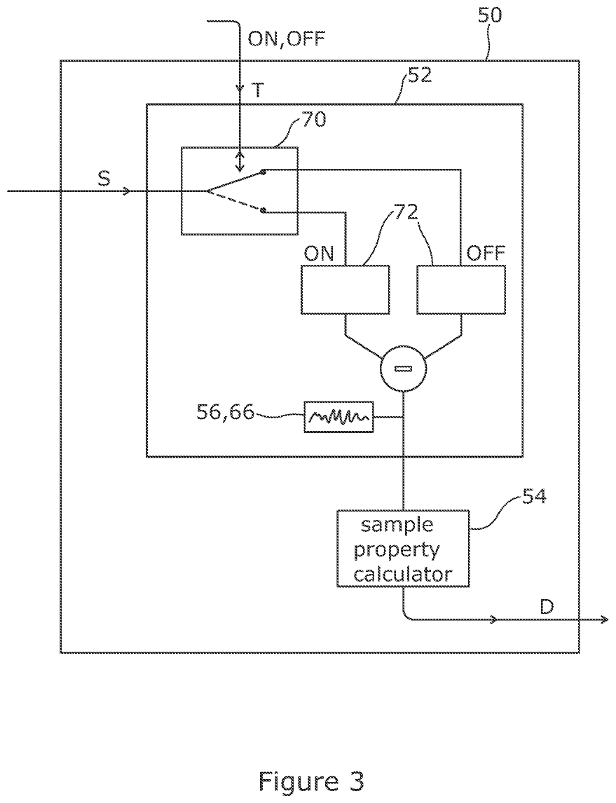

FIG. 3 illustrates another way in which the synchrony detector 52 may be implemented. In this example, the Raman spectral features S again take the form of a time series of Raman spectra received from the spectral detector 30 of FIG. 1. These are passed to a switch 70 which acts to direct each spectrum to one of two integrators 72 according to a binary state of the timing signal S, which could for example represent an ultrasound stimulus being either on or off, or some other pair of states of the stimulus. Raman spectra acquired while the timing signal is in a first state, for example “ON”, are therefore integrated together over time by a first of the integrators 72, while Raman spectra acquired while the timing signal is in a second state, for example “OFF”, are integrated together over time in a second of the integrators 72. The synchrony detector 52 then subtracts one integrated spectrum from the other to yield a difference spectrum which is output as the component spectrum 66 or synchronous features 56 which represent variations in the collected Raman scattered elements which have been induced by variations in the stimulus. This different spectrum can then be analysed as discussed above by the sample property calculator 54.

Although a repeated on-off cycle may be used, repeating a few or many times to build up the integrated spectra, from the arrangement of FIG. 3 it is also apparent how a single On-Off or Off-On cycle of stimulus variation can be used to determine variations in the Raman spectral features which are induced by variations in the stimulus. Detection of properties of the sample can be carried out using a single first interval during which the stimulus is on and in which Raman spectra are integrated into one of the integrators, and a single second interval during which the stimulus is off and in which Raman spectra are integrated into the other of the integrators (of course the two intervals can be swapped). Following just these two intervals, the spectral subtraction can be carried out to arrive at the difference spectrum, which represents changes in the Raman spectral features induced by variations in the stimulus. A multiplicative scaling factor can optionally be applied to one of the integrated spectra to provide better subtraction of the background, sample matrix, signal. This feature may be particularly useful in situations where on and off signal intensities from the sample matrix are not perfectly balanced.

Note that, although in FIG. 3 the component spectrum represents a difference between the measured Raman scattered elements when the stimulus in a first state, for example an “on” state, and the measured Raman scattered elements when the stimulus in a second state, for example an “off” state, it could instead represents a correlation between a larger set of states or continuously varying state of the stimulus and the measured Raman scattered elements.

The use of principal component analysis, partial least squares analysis, other multivariate methods, spectral subtraction, or various other spectral methods, to provide a component spectrum 66 from which to calculate properties of the sample properties enables good use to be made of the spectral data across a wide range of wavenumbers and spectral features such as spectral peaks in determining the required sample properties. However, other statistical techniques could be used to determine the sample properties from the Raman spectral features S such as automated machine learning techniques, or techniques which use much more limited Raman spectral features S such as data relating more specifically to one or more particular spectral lines or peaks.

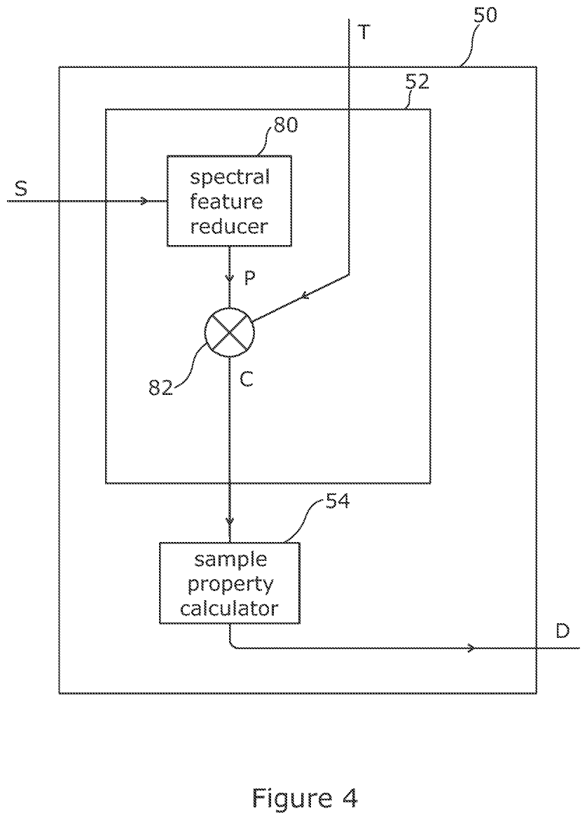

In the arrangement of FIG. 4 a different approach is used in which the synchrony detector 52 instead comprises a spectral feature reducer 80 and a correlator element 82. The spectral feature reducer 80 serves to reduce the dimensionality of the Raman spectral features S received from the spectral detector for example to a single time series spectral parameter P. This parameter P time series is then correlated using correlator element 82 with the timing signal T to provide a measure of correlation C between the two which is passed to the sample property calculator 54 to determine one or more properties of the sample.

The spectral feature reducer 80 may for example determine the contribution of a particular, predefined principal component or partial least squares component to each spectrum in a time series of spectra received from the spectral detector, this contribution time series then being used as the spectral parameter P. The parameter P may similarly be any parameter arranged to represent particular properties of the Raman spectra which are expected both to vary in a synchronous manner with the stimulus, and to enable target properties of the sample to be determined.

The correlator element 82 may use a cross-correlation or sliding dot product type calculation to determine a correlation between the parameter P and the timing signal T. The temporal correlation for some time span of P and T could be expressed as a single value, or could be output as a function C(τ) of a lag or time difference r between the two signals, where the lag might range over approximately the time taken to complete a single cycle of variation of the imposed stimulus. Of course, whether output as a single value or as a function of lag, the temporal correlation could be output either for a single time span, or as a function of time over multiple time spans. For example, in order to measure the progression over time of the concentration of SERS probes in a particular state, updated values or lag functions of C could be output at regular periods for example every minute.

Before the spectral property P and timing signal Tare received at correlator element 82, either or both may be subject to filtering or other modification, for example using long pass filters, where such filtering or modification is provided to improve the output of the sample property calculator.

The temporal correlation C is then passed to the sample property calculator 54 which determines from this signal the one or more properties of the sub-surface volume of the sample. The results D of this detection could for example either be provided as a binary indication of the presence of the SERS probes (which could be the SERS probes in a particular state such as a bound state with a target biomolecule), or could be an indication which also represents a particular level or concentration of such SERS probes.

For example, the sample property calculator could simply determine if any lag of the temporal correlation C(τ) exceeds a particular magnitude threshold, and if so then the SERS probes are found to be present. Alternatively, the sample property calculator could determine if a peak in the temporal correlation C(τ) has a sufficient finesse, such as a sufficiently narrow full width at half maximum measure, or other measure of quality, and if so then the SERS probes are found to be present. Instead or as well, the sample property calculator could seek a peak or other feature in the temporal correlation C(t) at a particular time lag t or in a particular range of time lags between the spectral property and timing signals.

Similarly, a particular level or concentration of the SERS probes (which could be the SERS probes in a particular state such as bound to a target molecule) could be derived by the sample property calculator from a magnitude, finesse, narrowness, of other quality of a peak or other feature in the correlation C(t), or such a property or combination of properties at a particular time lag or range of time lags.

Regardless of the precise techniques used to determine the sample properties D, these may then be used by the apparatus in various ways, for example for output on a display, for sounding or indicating an alarm if the properties D are outside some normal range, and may be stored in a local data store for example for comparison with future values. The detection results may also or instead be passed to other computer systems for example over a data network for storage, display and other purposes. Some embodiments may determine synchronous features 56 or a component spectrum 66 representing variations in the Raman spectral features S which are induced by the variations in the stimulus, and provide such synchronous features 56 or component spectra 66 as output rather than going on to determine properties of the sample. Such outputs can then be used at a later time by a practitioner or other user to look for or study the sample, the SERS probes, or other aspects of the sample system.

Although the analyser 50 is variously illustrated in FIGS. 1 to 4 as comprising a number of discrete functional elements, in practice these may be implemented together in software executing on one or more suitable computer systems. Aspects of the stimulation controller 42 and the spectral detector 30 may also be included in such software if appropriate. For example, the timing signal T may originate in software, and then be converted into a suitable analogue or digital signal for passing to the stimulation source 40, and the spectral detector 30 may process in such software raw optical detector, for example CCD, outputs, before passing to the spectral feature reducer 52.

Especially if the sample 14 is diffusely scattering, the proportion of scattering of the probe light within the sample which is inelastic Raman scattering, compared with the proportion of scattering which is elastic scattering, is typically very small, usually with a difference of many orders of magnitude, and especially when the sample is highly scattering as is typically the case with human or animal tissue and many other application areas. The SERS probes 10 will typically be found at rather low concentrations, so that the degree of Raman scattering from these will also be very small as a total proportion of scattering, even with the very large scattering cross section enhancements offered by the SERS process.

As a consequence, very few of the photons of probe light initially arriving at the sample will be Raman scattered in the sub-surface volume 12 or at the SERS probes 10. However, nearly every photon of probe light which is Raman scattered within the sub-surface volume or more specifically at the SERS probes 10 will also subsequently be elastically scattered a large number of times in that region and in the surrounding material of the sample 14, giving rise to a random walk of such photons before some emerge from the collection region 26 of the surface 16 to be collected by the collection optics 28.

The average path of this random walk through the sub-surface volume 12 and the surrounding sample 14, between the entry region 24 and the collection region 26, depends on the spatial offset between or distance between or relative geometry of these regions. It can be seen that for larger spatial offsets between entry and collection the average depth of the path will be deeper within the tissues.

Although in some embodiments, the entry and collection regions may be coincident or overlapping, using the above principle the spacing between the entry and collection regions can be controlled or adjusted by the apparatus 5, for example using an offset driver 25 depicted schematically in FIG. 1, in order to control the distribution of depths over which detection of Raman scattering within the sample and more specifically at any SERS probes occurs. This technique is referred to as spatially offset Raman spectroscopy (SORS), and is discussed in detail in WO2006/061565 and WO2006/061566, the contents of which are incorporated herein by reference for all purposes, including for illustrating how Raman features, or in the present case SERS probes in particular, may be detected at particular depths and profiles of depth within the sample. Essentially, by repeating the use of a varying stimulus with the entry and collection regions offset by multiple different spacings, a detection of the properties of the sample through induced variations of the spectral responses of the SERS probes can be carried out for each offset spacing and therefore for each corresponding depth or range of depths.

According to the principles of spatially offset Raman spectroscopy, the entry and collection regions may be of various sizes and shapes, and for any particular spatial offset these regions may each be formed by single contiguous or multiple discrete segments on the surface of the sample. For example, an entry region 22 may be provided at a fixed position, and multiple collection regions 26 may be provided at increasing spatial offsets from the single entry region, or a single fixed collection region and multiple entry regions could be used. However, in order to provide a stable and consistent provision of the imposed stimulus to the sub-surface volume, it may be desirable for both the entry and collection regions to move symmetrically with respect to the sub-surface volume 12.

The degree of elastic scattering within the diffusely scattering sample may vary depending on the nature of the sample, and may be defined in terms of transport length which is a length over which the direction of propagation of photon of probe light is randomized. The skilled person knows that transport length l* of diffusive scattering may be taken as being related to the mean free path by the expression:

$${l}^{*} = \frac{l}{1 - g}$$

where g is the asymmetry coefficient (average of the scattering angle over a large number of scattering events), and I is the mean free path. The diffuse scattering transport length for tissues of human or animal subjects for example, which may form the diffusely scattering sample of the present invention, may typically be of the order of 1 mm. More generally, the diffusely scattering sample 14 of the described embodiments may have a diffuse scattering transport length of less than 4 mm or less than 2 mm.

The one or more spatial offsets between the entry and collection regions used in embodiments of the invention may typically lie in a range of about 1 mm to about 50 mm, and more typically from about 3 mm to about 20 mm, and may be suitable for detecting properties of the sample in the sub-surface volume at depths beneath the surface of the overlying sample in the range from about 1 mm to about 80 mm and more typically from about 3 mm to about 50 mm. Embodiments of the invention may be arranged to detect properties of the sample at just one depth or depth profile, for example using a single spatial offset between the entry and collection regions, or may be arranged to detect properties at each of multiple depths or depth profiles. Embodiments may also use a zero or null offset in order to additionally detect properties of the sample at or near the surface of the overlying tissue.

FIG. 1 depicts entry and collection regions which are adjacent, proximal, or spaced apart on a surface of the sample which is largely planar or only moderately curved. Such an arrangement may be described as a backscatter configuration, because after penetrating into the sample and undergoing Raman scattering from SERS probes in the sub-surface volume 12, a photon of probe light is backscattered to the collection region 26 of the surface 16 for collection by the collection optics 28. However, the entry and collection regions may also lie on parts of the surface 16 which are far from coplanar, with substantially different surface normals, for example with normals in the region of 90 degrees apart, or even in the region of 180 degrees apart, or any other angle or range of angles.

For example, the entry and collection regions may be disposed on opposite sides of the sub-surface volume, or such that a sub-surface volume where properties of the sample are to be detected lies between the entry and collection regions, and such arrangements may be described as transmission configurations, noting that transmission configurations may also refer to geometries where the entry and collection regions are at other mutual angles relative to the sub-surface volume, for example around 90 or 135 degrees instead of around 180 degrees in angle.

Transmission arrangements in which the sub-surface volume 12 lies directly or approximately between the entry and collection regions may be of particular interest where the sample 14 is accessible from both side and its thickness is of the order of about 5 mm to about 80 mm, although larger or spacings between entry and collection regions could be used if required. As for the SORS arrangements described above, the SERS probes may conveniently be located at distances beneath the surface of the sample which range from about 1 mm to 80 mm, of from about 3 mm to 30 mm, or may be located throughout the sample or distributed in other ways.

Further discussion of transmission geometries and other details of such arrangements which can be used in embodiments of the present invention, to detect properties of a sub-surface volume of a diffusely scattering sample, can be found in the prior art including WO2007/113566, the contents of which is incorporated herein by reference in its entirety, to demonstrate how to arrange suitable transmission geometries for use in the present invention, and for all other purposes.

In FIG. 1 the stimulation source 40 is generally shown as directing the imposed stimulus towards the surface of the sample proximally to the sub-surface volume 12 where SERS probes 10 are to be detected, and in some embodiments, all or a large portion of the sample and an extended sub-surface volume 12 may be exposed to the varying stimulus at the same time, for the purposes of detecting the SERS probes over a large sub-surface volume 12 from which scattered light is collected by the collection optics 28.

However, the extent of the sub-surface volume 12 in which properties of the sample are to be detected may be controlled or restricted by one or both of the geometry of the delivery and collection optics, and the geometry of the stimulus within the sample 14.

In some embodiments therefore, the stimulation source 40 is arranged to limit the spatial extent of the stimulus within the sample so that the sub-surface volume 12 within which the SERS probes are responsive to the stimulus is correspondingly limited. The particular ways in which this can best be achieved will vary according to the type of imposed stimulus, as discussed further below, but generally speaking the volume of the sample within which the SERS probes are responsive to the stimulus may be limited, by the spatial extent of the stimulus within the sample, for example to no more than 1000 mm³ or no more than 100 mm³ or no more than 10 mm³ or no more than 1 mm³.

Similarly, in order to achieve such a similar limitation, the volume of the sample within which the stimulus is greater than half, or greater than 1/e, or greater than 1/10 of its maximum or peak value within that volume at the same moment in time, may be limited to no more than 1000 mm³ or no more than 100 mm³ or no more than 10 mm³ or no more than 1 mm³.

If the imposed stimulus is ultrasound, for example, then such volumes can easily be achieved using suitable focussing ultrasound transducers. If the imposed stimulus comprises magnetic fields, then such volumes can easily be achieved by using suitably shaped magnets, and if the imposed stimulus is light then such volumes can be achieved by suitable choice of wavelength(s), limited spot size at the sample surface, and appropriate depths of the sub-surface volume 12 within which the SERS probes are to be responsive to the stimulus.

The apparatus of FIG. 1 may also be arranged to scan across the sample, through relative movement of the sample and/or the relevant optics, by repeating the detection of properties of the sample for a plurality of separate or overlapping sub-surface volumes 12 within the diffusely scattering sample 14, for example with the sub-surface volumes forming a line or grid of locations within or across the sample. In this way, a section or map of the detected SERS probes can be formed, typically in one or two dimensions across the surface of the sample, but optionally in three dimensions including depth within the sample if required. If a line section is scanned then this could be a straight line, a circle or some other form. If a two dimensional grid is scanned then this could be a rectilinear, hexagonal or some other grid form.

This scanning can be achieved by directing the stimulus to each of the plurality of sub-surface volumes in turn, for sufficient time at each such volume for the properties of the sample to be detected using the techniques described above. As described above, a stimulation source 40 may be used or controlled such that the stimulation is focussed to each sub-surface volume.

If the range of the scanning or mapping is sufficiently small relative to the spacing between and sizes of the delivery and collection optics, then the scanning action could be implemented holding the stimulation source stationary but controlled to move the focus of the imposed stimulation to each required sub-surface volume in turn. This might be suitable for example if a 10×10 grid of 1 mm³ sub-surface volumes are scanned in turn by electronic control of an ultrasound stimulation source. If the required range of scanning is larger then it may be desirable to physically translate one or both of the stimulation source, and the combination of the delivery and collection optics, or alternatively to translate the diffusely scattering sample itself.

The SERS probes may take a number of different forms, but typically may comprise metal nanoparticles, and more particularly noble metal nanoparticles for example of silver, gold or platinum, and typically having diameters approximately in the range of 10 nm to 500 nm. The SERS probes may further comprise one or more Raman reporter molecules bound to the surface of such nanoparticles.

In the particular experiment described below, the SERS probes were gold nanoparticles of approximately 60 nm in diameter, coated with biphenyl-4-thiol Raman reporter molecules adsorbed onto the metal surfaces. These SERS probes are demonstrated in the experiment described below to display a Raman spectral response which is spectrally responsive to an ultrasound stimulus, which is thought to occur through changes in temperature at the SERS probes driven by the ultrasound stimulus.

Various other SERS probes which are suitable for the present purposes are described in the literature, for example see: Langer J. et al., “Present and future of surface—enhanced Raman scattering”, ACS Nano 2020, 14, 1, 28-117; Perumal J. et al., “Towards a point-of-care SERS sensor for biomedical and agri-food analysis applications: a review of recent advancements”, Nanoscale, 2, 2021; and Pilot, R. “A review on surface-enhanced Raman scattering”, Biosensors 2019, 9, 57.

Instead of using nanoparticle probes, the SERS probes may instead be provided by larger substrate or particles having suitably prepared, e.g. roughened, surfaces so as to provide enhanced Raman effects. For example, microscopic to macroscopic structures may be provided with a metal surface which is patterned or etched to provide a SERS effects at particular points to which Raman reporter molecules may be bound if desired. For example, see Langer J. et al. cited above.

A variety of different types of varying stimulation, and typically time varying stimulation may be used, either alone or in combination with each other. In the particular experiment described below, an ultrasound source is used as the stimulation source 40 to deliver a time varying stimulation to the sub-surface volume. Both focussed and unfocussed ultrasound waves can be used as an effective time varying stimulus. Typically, these can be delivered using an ultrasound transducer as the stimulation source 40. A convenient ultrasound device to use is a high-intensity focussed ultrasound (HIFU) transducer, widely used in prior art medical treatments. Conveniently, HIFU transducers are available in a ring form with a central opening or aperture, and the delivery and collection optics and/or the entry and collection regions of FIG. 1 can then be located within that central opening. See for example the HIFU Plex transducer bundles provided by Verasonics, Inc. of Kirkland, WA, USA.

Some HIFU transducers can provide a focal spot with a diameter of the order of 1 mm, or even 0.1 mm, and therefore using such a transducer the described techniques can be used to modify the spectral response of SERS probes within a sub-surface volume of comparable size. A discussion of spatial and temporal resolutions which can be achieved by such transducers is found in Alexander and Swanevelder, “Resolution in ultrasound imaging”, Continuing Education in Anaesthesia, Critical Care & Pain, Volume 11, Number 5, 2011, also found at https://doi.org/10.1093/bjaceaccp/mkr030.

The spectral response of SERS probes to an ultrasound stimulus may be in the form of spectral shifts or intensity changes in existing features such as Raman spectral peaks of reporter molecules bound to the SERS probes, or in conformational changes leading to the appearance of new features or the disappearance of existing features. These spectral responses may be due to temperature changes within the bulk sample, temperature changes induced at the SERS probes themselves, or due more directly to mechanical changes at the SERS probes induced by the ultrasound.

A time variation of the ultrasound stimulus within the sub-surface volume may be for example in the form of a square, sinusoidal, triangular, or other form of intensity variation, with a cycle time which is suitable for driving sufficient spectral responses of the SERS probes. For example, if a larger volume of a few cubic centimetres is being stimulated then a cycle time of a few seconds may be appropriate, but a much shorter cycle time of say a tenth of a second might be used for a much smaller volume. Although the time variation of the stimulation may take the form of a regular, repeating cycle, this is not necessary for a temporal correlation between a time varying stimulus and the spectral response of the SERS probes to be measured and used for detection of properties of the sample.

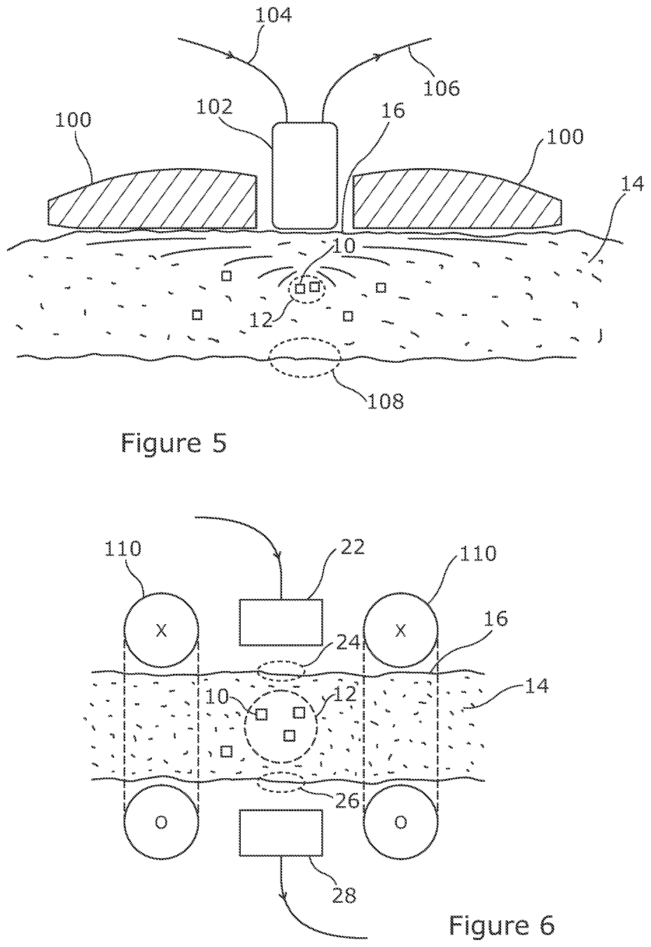

FIG. 5 illustrates one way in which the stimulation source and the delivery and collection optics of FIG. 1 may be implemented if ultrasound is being used for the varying stimulation. The depicted stimulation source is a HIFU ultrasound transducer 100 which provides a focussed ultrasound region which defines the sub-surface volume 12 within which SERS probes 10 are to be responsive. A Raman probe 102 combines the delivery and collection optics, for example with a first bundle of optical fibres 104 delivering the probe light to the Raman probe 102 and a second bundle of optical fibres 106 carrying the collected light to the spectral detector 30 of FIG. 1. Typically, the entry region 24 may be defined by the first bundle of optical fibres 104 coming into close contact with the sample 14 in a central region of the end portion of the Raman probe. Concentric rings of the second bundle of optical fibres 106 terminating in close contact with the sample may then provide discrete collection regions 26 at multiple spatial offsets from the central entry region, or vice versa.

Because the SERS probes are spectrally responsive only where the time varying ultrasound stimulus is of sufficient intensity to give rise to suitable variations in their spectral response, only SERS probes 10 that are within the focal region of the ultrasound give rise to a spectral response induced by the stimulus. Since the ultrasound can be focussed to very small regions, high spatial resolution, for example better than 1 mm, can be obtained for detecting an induced response of the SERS probes and corresponding detection of sample properties. Moreover, the HIFU ultrasound transducer can be controlled to scan the focal point of the ultrasound over time, so that SERS probes at multiple points of a line or grid can be detected in turn, and without any relative physical movement of the transducer or Raman probe.

In a variation of the arrangement of FIG. 5, delivery optics 22 are used to direct probe light to an entry region 24 on the sample located in the centre of the ultrasound probe 100, and collection optics are then used to collect probe light which has been scattered through the sample 14 to a collection region 108 disposed on the opposite side of the sample, which may be referred to as a transmission or forward scattering geometry.

Another type of time varying stimulation that may be used is a time varying, or oscillating, or alternating (AC) magnetic field, typically provided by one or more electrical coils. The SERS probes are then required to exhibit a spectral response to the magnetic field stimulus, for example in the form of spectral shifts or intensity changes in existing features such as Raman spectral peaks of reporter molecules bound to the SERS probes, or in conformational changes leading to the appearance of new features or the disappearance of existing features. These effects may take place by direct mechanical perturbation of the SERS probes through mechanical translation, rotation, vibration, or conformation or shape change due to the magnetic field, thermal heating of the SERS probes and/or the surrounding sample material, the stripping of Raman reporter molecules, or in other ways.

Such a magnetic stimulus will typically be in the form of an oscillating, or AC magnetic field, for example with an AC frequency of some kHz of tens of kHz, where the variation in the magnetic field which is applied to induce detectable variations in the measured Raman scattered elements may be a variation in that AC field with a time period of the order of 0.001 to 100 seconds.

Typically, in order for such effects to take place the SERS probes may be in the form of magnetic nanoparticles, which may for example comprise a core of a magnetic material, surrounded with a noble metal layer, and on to which Raman reporter molecules are bound. Some examples of such magnetic SERS nanoparticles are found for example in Wang et al., “Magnetic plasmonic particles for SERS-based bacteria sensing: A review”, AIP Advances 9, 010701 (2019), also found at https://doi.org/10.1063/1.5050858. Other examples are found in Song et al., “Applications of magnetic nanoparticles in surface-enhanced Raman scattering (SERS) detection of environmental pollutants”, Journal of Environmental Sciences 80, 2019, pages 14-34, also found at https://doi.org/10.1016/i.jes.2018.07.004.

A time variation of the magnetic stimulus within the sub-surface volume may be similar to that for ultrasound, for example in the form of a square, sinusoidal, triangular, or other form of envelope in magnetic field strength variation applied to the underlying typically AC magnetic field, with a cycle time which is suitable for driving sufficient spectral responses of the SERS probes. However, as well as or instead of intensity variations, the magnetic field may be driven with variations in direction, for example oscillating in a linear direction and/or rotationally, and in other ways. Typically it will be difficult to provide as small a focal point with a magnetic field as with ultrasound, but detection of SERS probe response within a sub-surface volume of a few hundred to a few thousand cubic millimetres or larger should usually be possible. A cycle time of the time variation of a few seconds may be appropriate.

FIG. 6 illustrates one way in which the stimulation source and the delivery and collection optics of FIG. 1 may be implemented if a magnetic field is being used for the time varying stimulation. The depicted stimulation source takes the form of a pair of electrical coils 110 which in this case provide a reasonably homogenous magnetic field within the sample 14, and in particular within a sub-surface volume 12 within which SERS probes 10 are to be detected. In this figure, delivery optics 22 and collection optics 28 are disposed on either side of the sample in a transmission geometry.

In variations of the arrangement of FIG. 6, a magnetic field which is much more focussed on a particular smaller volume of the sample 14 may be provided, and/or the electrical coil or coils could be disposed only to one side or face of the sample.

Another type of time varying stimulation that may be used is a time varying field of light or another type of electromagnetic radiation. For example, laser light may be used. In a diffusely scattering sample the ability to restrict the stimulating light to a small sub-surface volume is limited, but may be used to some extent for example to provide a sub-surface volume for sample property detection which is about the same size as or a bit larger than the maximum depth of the volume to be detected below the surface. The wavelength of the stimulus light may be chosen to be in a band where the sample is non or only weakly absorbing, and ideally non-overlapping with either the probe light as directed into the sample, or portions of the Raman spectra of the SERS probes which are to be used for temporal correlation with the time varying light stimulus.

SERS probes may be provided which exhibit a spectral response to the time varying light stimulus in various ways, for example by the stimulus light being absorbed at the SERS probes or in the surrounding sample leading to heating and related spectral responses of the reporter molecules (for example by inducing spectral shifts to Raman bands or by switching the reporter molecules to a different conformation). If the SERS probes are in the form of nanorods, such as gold nanorods then the light stimulus at an absorption wavelength of the nanorods may be used to reshape the nanorods and thereby lead to a related spectral response of Raman reporter molecules bound to the nanorods.

The form of the time variation of a light stimulus may be similar to those discussed above in respect of ultrasound and magnetic field stimuli, for example in the form of an on-off square wave, or sinusoid of intensity, with a cycle time of a few seconds.

Another type of varying stimulation that may be used is a time varying field of microwaves or terahertz waves, for example in a wavelength range from about 0.1 mm to 1000 mm. At the shorter wavelength end of this spectrum, with higher frequency microwaves and terahertz radiation, it is possible to provide a sub-surface volume for response of SERS probes with a diameter of the order of a few mm or less, noting that the level of diffusive scattering of microwaves and terahertz waves in typical diffusely scattering media tends to be much less than that of visible light, thus enabling a small and reasonably well defined sub-surface volume for detection of sample properties to be defined.

The microwave or terahertz wave stimulus may cause a spectral response in the SERS probes for example by very localised heating at the SERS probes themselves and/or in the wider sample, or by more direct interactions with the Raman reporter molecules themselves.

The form of the time variation of a microwave or terahertz stimulus may be similar to those discussed above in respect of ultrasound, magnetic field, and light stimuli, for example in the form of an on-off square wave, or sinusoid of intensity, with a cycle time of a few seconds.

Another type of time varying stimulation that may be used is a time varying thermal field, typically caused by direct thermal coupling into the sample rather than heating using another stimulus which penetrates into the sample. For example, the sample could be heated in a time varying manner by infrared radiation directed to the surface of the sample, or by immersion in a flow or thermal bath of water or another fluid the temperature of which varies according to a predefined or controlled cycle. The rate of spectral response of the SERS probes to such thermal stimulation is likely to be rather slow when compared to other stimulus methods discussed above, due to the need for the heat to conduct into the sample, but for smaller samples this technique may be very effective, and may be attractive for both its simplicity of execution, and for avoiding the incidence of potentially damaging radiation to a more delicate sample.

In embodiments of the invention, SERS probes may be used to detect sample properties in a wide range of diffusely scattering samples and for a wide range of purposes. For example, if the sample is a portion of a human or animal body then suitable SERS probes which are spectrally responsive to a stimulus as discussed above may be used for cancer diagnosis, or for the monitoring or measurement of:
neuro transmitters in vivo
muscle physiology during exercise to evaluate fatigue
inflammation
bone turnover for example using biophosphonate labelled nanoparticles
disease using anti-bodies, for example Alzheimer's disease, infectious agents, or fungal uptake in lungs
progression of treatments such as tissue healing, cancer treatments, tissue regeneration (for example of bone scaffolds)
biomedical conditions via monitoring of bioanalytes such as glucose
regenerative tissue and treatment progress, for example with measurements specific to cell type and local environment.

Such application areas may include detection of sample properties using suitable SERS probes in a wide variety of in-vivo body portions, for example external detection through the skin of the head, neck, trunk, or various limbs. Such application areas may also include detection of sample properties using suitable SERS probes through surgically exposed surfaces of the body, or surfaces accessed using suitable endoscope equipment such as the within the mouth, nose, larynx, stomach, or intestines.

Other samples within which the spectral response of SERS probes to stimulus may be used for property detection may occur in biopharma and quality monitoring processes, the sensing of colloidal systems, the imaging of plants, and within various manufacturing processes such as the manufacture of polymers, in each case utilising either or both freely floating SERS probes or immobile SERS probes (e.g. a SERS substrate attached to a bioreactor wall).

Although the embodiments described above have generally related to detecting properties using SERS probes 10 within diffusely scattering samples 14, the invention can also be applied to other situations where no, or minimal, diffusely scattering material lies between the SERS probes to be detected and the collection optics arranged to collect light from the SERS probes for measurement of the Raman scattered elements. Some examples of suitable application areas include those discussed in Fan et al., “A review on recent advances in the applications of surface-enhanced Raman scattering in analytical chemistry”, Analytica Chimica Acta 1097, pages 1-29, February 2020. For example, the SERS probes discussed herein may be integrated into fluidic or microfluidic systems, sample cells, optical fibres, lateral flow tests, and similar structures.

In such examples, the SERS probes do not necessarily reside in a sub-surface volume, and may instead be at or close to a surface of a sample or corresponding structure carrying the SERS probes, and/or may be located within or behind a transparent, or largely transparent, structure or substrate.

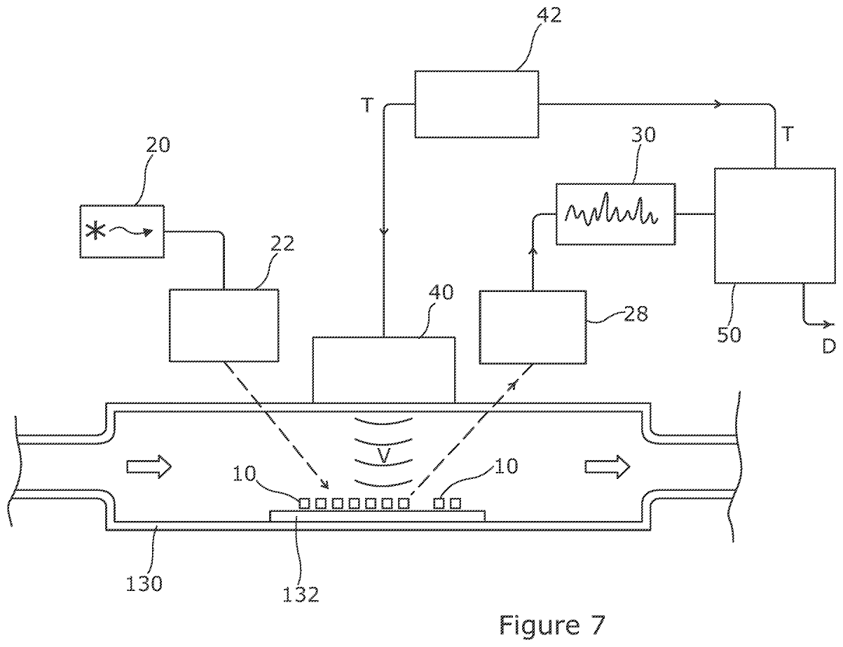

By way of example, FIG. 7 illustrates a flow cell 130 through which a sample fluid may be passed. SERS probes 10 which are spectrally responsive to a stimulus, as variously discussed elsewhere in this document, may be bonded to, or secured to, or form part of, a substrate 132, in a manner so as to be exposed to the sample fluid flowing through the cell. Delivery optics 22 then direct probe light, such as laser light as discussed elsewhere in this document, to the SERS probes 10 on the substrate, and collection optics 28 collect probe light scattered from the substrate 132, which includes elements of the probe light Raman scattered from the SERS probes 10. The probe light may be directed into and collected from the flow cell through one or more transparent walls of the flow cell 130, or through transparent windows in those walls, or in other ways.

Chemical species or other targets in the fluid flowing through the sample cell 130 are then able to bind to Raman active molecules of the SERS probes to thereby change the Raman spectral response and particular Raman scattered elements of the probe light measured and output by the spectrometer 30 as Raman spectral features S.

The stimulation source 40 as already discussed elsewhere in this document then provides a stimulus in varying form, typically a time varying form, to the SERS probes, under control from a stimulation controller 42 for example using a timing signal T, leading to corresponding variations in the measured Raman scattered elements of the probe light, and corresponding variations in the Raman spectral features S. The analyser 50 then detects properties of the sample of the SERS probes from variations in the measured Raman scattered elements which are induced by the variations in the stimulus, for example using principal component analysis and correlation, or other techniques, for example as already discussed above, for output as detection results D. The detection of the properties of the sample may be a detection of the SERS probes themselves, for example a detection of the SERS probes in a particular state, such as bound to, or not bound to particular targets such as the chemical species or other targets to be detected in the fluid.

In the case of the flow cell 130 of FIG. 7, the fluid could for example be atmospheric air and the detection process provides the detection of particular chemical species or particles in the air such as environmental pollutants, or gasses themselves, such as gasses in industrial processes (e.g. biopharmaceutical, pharmaceutical, chemical, petrochemical). Alternatively the fluid could be breath of a human or animal subject in which certain species are to be detected. Alternatively, the fluid could be a liquid or gas in a biological or other manufacturing process, and the detection process provides detection of target chemical species or conditions such as pH in that fluid.

Of course, although the substrate 132 and SERS probes of FIG. 7 are illustrated as being present within a flow cell 130, they may instead be presented in other arrangements such as on a surface or within a substrate of a medical test such as a lateral flow test, within a microfluidics system, as part of an assay, or arranged to monitor catalytic reactions in various contexts.

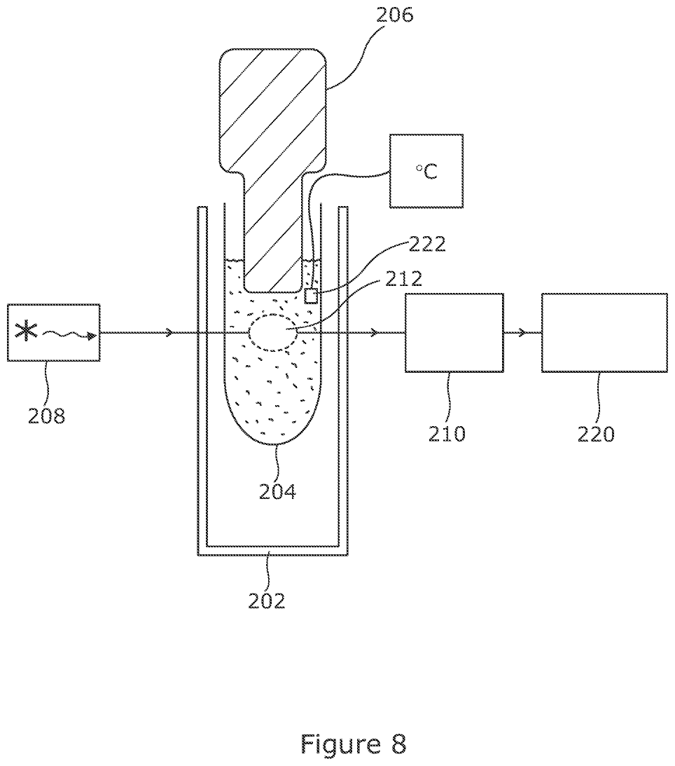

FIG. 8 illustrates the arrangement used for a particular experiment to demonstrate synchrony or correlation between a time varying stimulus, and corresponding temporal variation in the Raman spectral response of SERS probes exposed to that stimulus, which can therefore be used to implement embodiments of the invention. In particular, the experiment demonstrates that the spectral shape or form of the Raman spectral signal of SERS probes can be altered using an ultrasound stimulus, in a reversible manner. In this experiment a correlation is demonstrated between the score of a component spectrum of the measured Raman spectral features which is a selected principal component of the spectral variation over time, and the timing signal T.

A rectangular PMMA cuvette 202 of size 10×10×45 mm was used to hold a polypropylene micro-tube 204 containing a solution of SERS probes in the form of gold nanoparticles in 1 ml of water. The nanoparticles were 60 nm in diameter, coated with biphenyl-4-thiol (BPT) Raman reporter molecules. The tip of an ultrasound probe 206 was placed in the solution to provide an ultrasound field within the solution and at the SERS probes. The ultrasound probe 206 was a Fisher Scientific 50 Sonic Dismebrator with a 3 mm diameter titanium tip, operated at 20 kHz and 10 Watts.

A continuous wave laser 208 was used to generate probe light at a wavelength of 830 nm, and the probe light was directed to the micro-tube so as to enter the solution as a beam of 0.5 mm diameter and a power of 150 mW. Collection optics 210 were used to collect scattered light from the solution at an angle of 150 degrees with respect to the incident probe light beam, and over a collection region of 1.5 mm in diameter at the surface of the micro-tube. This was therefore a transmission geometry of collection, with the entry and collection regions on opposite sides of the sample, although not at exactly opposing points of the micro-tube. The passage of the probe light into the micro-tube and the collection of scattered light took place around a region of the solution 212 which was about 10 mm beneath the tip of the ultrasound probe 206.

The collected light was passed to a spectrometer 220 for detecting elements of the probe light Raman scattered from the SERS probes, and in particular Raman spectra over a spectral region of interest from 850 to 1700 cm⁻¹.

During the experiment, the ultrasound probe was turned on for 90 seconds, turned off for some minutes, then turned on for 150 seconds, off for some minutes, turned on for 370 seconds, and then off again for some minutes, with a total run time of 37.5 minutes. Raman spectra were collected for each sequential 5 second interval, by integrating the signal from the spectrometer 220 over that interval. In a separate experiment under the same conditions and timing parameters, the temperature of the solution was measured using a thermocouple 222.

After spectral calibration, baseline subtraction, and spectral smoothing, principal component analysis (PCA) was performed on the 450 Raman spectra using the “SOLO” analysis tool provided by Eigenvector Research Incorporated of Manson, WA, USA.

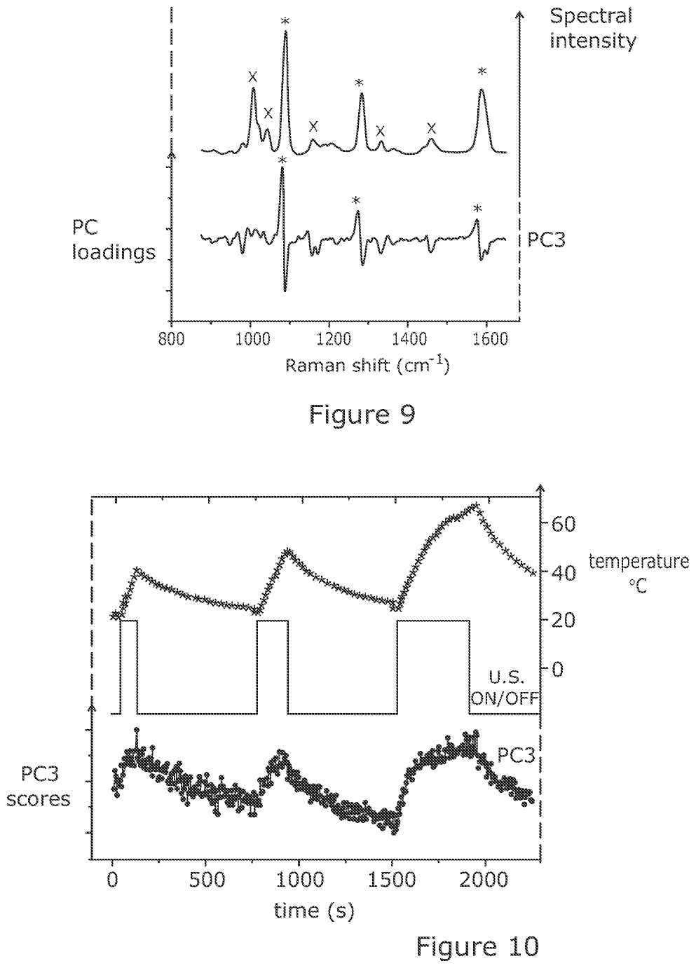

FIG. 9 shows, as the upper curve and right hand scale, an average SERS Raman spectrum acquired over the experiment. The “X” symbols mark spectral peaks characteristic of the PMMA cuvette and polypropylene micro-tube, and the asterisk symbols mark spectral peaks characteristic of the BPT Raman reporter molecules.

Principal component analysis of the 450 acquired spectra gave rise to three main principal components. The third of these, PC3, is plotted as the lower curve in FIG. 5 against the left hand scale, and resembles a first order derivative of the spectrum with respect to wavenumber, indicating frequency shifts in the corresponding Raman spectral features of the BPT molecules which are marked by the asterisks.

FIG. 10 then shows, as the upper curve and right hand scale, temperature of the solution measured using the thermocouple 222 during the experiment. A central curve of FIG. 10, without ordinate scale, shows the on/off cycle of the ultrasound stimulus. It can be seen that the temperature rises steeply during when the ultrasound stimulus is on, and falls away again more gradually when it is off.

The lower curve of FIG. 10 shows a score of the PC3 component indicating its contribution to the Raman spectra acquired in each time interval, with the scores represented by the left hand scale. It can be seen that the PC3 score tracks the solution temperature closely, and is also therefore strongly correlated with the timing signal of the ultrasound stimulus. The nature of the SERS spectral shift represented by the PC3 component and it's correlation with temperature is also consistent with temperature related anharmonic coupling of low frequency skeletal modes of the BPT molecule to high frequency vibrational modes.

The various aspects of the invention described above may be considered both in terms of methods and apparatus. Some aspects particularly relate to data processing, for example the synchrony detector 52 and sample property calculator 54 as depicted in FIGS. 1-4. Such aspects may typically be implemented using computer software executing on one or more computer systems, and if multiple computer systems are used these could be collocated within a single analyser unit or could be geographically or spatially distributed if required.

Such computer systems may typically comprise one or more microprocessors to execute computer program code implementing such aspects, and volatile and/or non-volatile memory to store the program code, input data such as spectral data received from the spectral detector, carry out the described processes, and output data such as detection or concentration of particular SERS probes or such SERS probes in one or more particular states. To this end, the invention also provides one or more computer readable media comprising computer program code arranged to carry out such aspects of the invention.

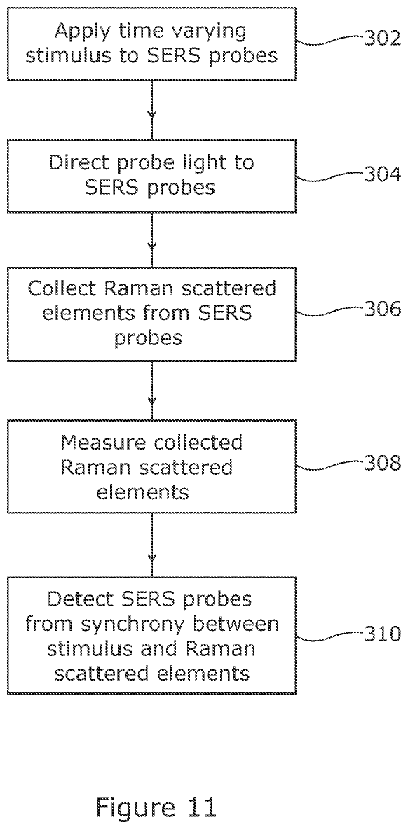

FIG. 11 illustrates methods of the invention already described above in detail but now using a flow chart form. In step 302 a stimulus such as an ultrasound, magnetic, light, other electromagnetic, or thermal stimulus is applied to SERS probes in a time varying form, typically using a suitable stimulation source which gives rise to corresponding changes to the spectral shape or form of Raman spectra arising from the SERS probes. As discussed above, the SERS probes may be embedded or contained within a diffusively scattering sample, or may be exposed as a suitable surface. Also as noted above, the time variation of the stimulus could take various different forms such as a cycle repeated many times, or in some case as little as a single cycle of Off-On or On-Off.

In step 304, during the application of the time varying stimulus, probe light, typically laser light, is directed to the SERS probes. If the SERS probes are embedded within a diffusely scattering sample then the probe light arrives at the SERS probes by diffusive transport through the sample. The probe light may typically be in the form of a pulsed or constant laser beam, but will typically not vary in synchrony with the time varying stimulus but instead will usually be substantially constant on the time scales of that time variation.

In step 306, portion of the probe light is collected, including elements of said probe light which have been Raman scattered from the SERS probes. Since Raman scattering is a process with a low cross section, most of the collected probe light will have been scattered from the SERS probes and surrounding sample or other structures or media without any Raman scattering having taken place, but these elastically scattered elements can largely be removed from the collected light using suitable optical filtering. If the SERS probes are embedded within a diffusely scattering sample, then the Raman and other probe light will be collected following multiple elastic scattering events in the sample, and typically from a collection region on the sample which is suitably spaced from the entry region at which the probe light was directed into the sample.

As step 308 the collected Raman scattered elements are measured for example using a spectrometer, and at step 310 one or more properties of the SERS probes or surrounding sample are detected based on variations in the measured Raman scattered elements which are induced by the stimulus, for example from synchrony or temporal correlation between the applied time varying stimulus and corresponding temporal variations in the measured Raman scattered elements of the probe light. As discussed above, this detection may be carried out in various ways for example by selecting a principal component or partial least squares component of the time series of Raman spectra which is best correlated with the variations in the applied stimulus.

Although particular embodiments and applications of the invention have been described, it will be apparent to the skilled person that various modifications and alterations can be made without departing from the scope of the invention.

1. A method of detecting one or more properties of a sub-surface volume of a diffusely scattering sample which comprises surface enhanced Raman Spectroscopy (SERS) probes, the SERS probes being spectrally responsive to a stimulus, the method comprising:
providing the stimulus in a varying form at the sub-surface volume;
during the varying stimulus, directing probe light to an entry region on a surface of the sample, and collecting a portion of the probe light, including elements of said probe light Raman scattered from said SERS probes, at a collection region on the surface of the sample, the collection region being spaced from the entry region;
measuring the collected Raman scattered elements;
detecting variations in the measured Raman scattered elements which are induced by the variations in the stimulus; and
detecting the one or more properties based on the detected variations in the measured Raman scattered elements.

2. The method of claim 1 wherein the variations in the stimulus comprise changes in the stimulus over time at the sub-surface volume, and the variations in the measured Raman scattered elements which are induced by the variations in the stimulus comprise changes in the measured Raman scattered elements over time.

3. The method of claim 1 wherein detecting the variations in the measured Raman scattered elements which are induced by variations in the stimulus comprises detecting such variations that are in synchrony with, or are correlated with, the variations in the stimulus.

4. The method of claim 1 wherein the variations in the measured Raman scattered elements induced by variations in the stimulus comprise variations in one or more of: the spectral shape or form, the intensity or relative intensity, the width, and the wavenumber, of one or more Raman spectral features of the measured Raman scattered elements.

5. The method of claim 1 wherein the step of detecting the one or more properties comprises forming a component spectrum representing variations in the measured Raman scattered elements induced by variations in the stimulus, and determining the one or more properties from the component spectrum.

6. The method of claim 5 wherein the component spectrum is a principal component of the measured Raman scattered elements, formed using principal component analysis, or a partial least squares component of the measured Raman scattered elements, formed using partial least squares analysis, the principal or partial least squares component representing changes in the measured Raman scattered elements which are induced by changes in the varying stimulus.

7. The method of claim 1 wherein the component spectrum represents correlation between the measured Raman scattered elements and the stimulus.

8. The method of claim 7 wherein the component spectrum represents a difference between the measured Raman scattered elements when the stimulus in a first state for example an “on” state, and the measured Raman scattered elements when the stimulus in a second state for example an “off” state.

9. The method of claim 1 wherein detecting the one or more properties based on the detected variations in the measured Raman scattered elements comprises detecting the SERS probes.

10. The method of claim 1 wherein the detecting the one or more properties comprises detecting one or more of:
particular states of the SERS probes;
binding states of the SERS probes to one or more target species;
a temperature at the SERS probes;
a pH at the SERS probes;
a density or quantity of the SERS probes; and
presence or absence of the SERS probes.

11. The method of claim 1 wherein each SERS probe comprises one or more metal nanoparticles, each metal nanoparticle carrying one or more Raman reporter molecules, wherein the elements of said probe light Raman scattered from the SERS probes are elements of the probe light scattered from the Raman reporter molecules.

12. The method of claim 1 wherein the varying stimulus is one or more of: a non-optical or non-light stimulus; an ultrasound stimulus; a magnetic stimulus; a time varying ultrasound field stimulus; a time varying magnetic field stimulus a time varying light field stimulus; and a time varying microwave or terahertz wave field stimulus.

13. The method of claim 1 wherein the varying stimulus is a time varying ultrasound field generated by a high-intensity focussed ultrasound transducer having a central aperture, and one or both of the entry region and collection region are located within or visible through the central aperture.

14. The method of claim 1 wherein a cycle time of the varying stimulus for inducing detected variations in the Raman scattered elements of the same or a corresponding cycle time, is between 0.001 and 100 seconds.

15. The method of claim 1 wherein the entry and collection regions are disposed on opposite sides of the sub-surface volume.

16. The method of claim 1 comprising separately detecting the SERS probes for each of a plurality of different spatial offsets between the entry and collection regions; associating the detected SERS probes for each different spatial offset with a different depth or distribution of depth within the sample; detecting the one or more properties at each different depth or distribution of depth based on the detected variations in the measured Raman scattered elements.

17. The method of claim 16 wherein the entry and collection regions are spatially offset by an offset in the range from 1 mm to 50 mm, and more preferably in the range from 3 mm to 20 mm.

18. The method of claim 1 wherein the SERS probes are located at distances beneath the surface of the sample which are in the ranges of: at least 1 mm; from 1 mm to 80 mm; and from 3 mm to 50 mm.

19. The method of claim 1 wherein the diffusely scattering sample has a diffuse scattering transport length of less than 4 mm.

20. The method of claim 1 wherein the stimulus is provided in the sub-surface volume such that a volume of the sample, within which the stimulus has a magnitude greater than 1/e of its corresponding peak spatial magnitude within that volume, or a volume of the sample in which the SERS probes can be detected, is limited to no more than 1000 mm³ or to no more than 100 mm³.

21. The method of claim 1 comprising repeating the method to detect the one or more properties at a plurality of different sub-surface volumes within the diffusely scattering sample, wherein for each such sub-surface volume the stimulus in varying form is provided to that sub-surface volume for the detection of the properties in that sub-surface volume.

22. The method of claim 1 wherein the sample is an in-vivo portion of a human or animal body, and optionally wherein the surface of the sample is skin of the in-vivo portion.

23. Apparatus for detecting one or more properties of a sub-surface volume of a diffusely scattering sample which comprises SERS probes, the SERS probes being spectrally responsive to a stimulus, the apparatus comprising:
a stimulation source arranged to provide the stimulus in a varying form at the sub-surface volume;
delivery optics arranged to direct probe light to an entry region on a surface of the sample during the varying stimulus, and collection optics arranged to collect a portion of the probe light, including elements of said probe light Raman scattered from said SERS probes, at a collection region on the surface of the sample, the collection region being spaced from the entry region;
a spectral detector arranged to measure the collected Raman scattered elements; and
an analyser arranged to detect variations in the measured Raman scattered elements which are induced by the variations in the stimulus, and to detect the one or more properties based on the detected variations in the measured Raman scattered elements.

24. The apparatus of claim 23 wherein the variations in the stimulus comprise changes in the stimulus over time at the sub-surface volume, and the variations in the measured Raman scattered elements which are induced by the variations in the stimulus comprise changes in the measured Raman scattered elements over time.

25. The apparatus of claim 23 wherein the analyser detects the variations in the measured Raman scattered elements which are induced by variations in the stimulus by detecting such variations that are in synchrony with, or are correlated with, the variations in the stimulus.

26. The apparatus of claim 23 wherein the variations in the measured Raman scattered elements induced by the stimulus comprise variations in one or more of the intensity, the width, and the wavenumber of one or more Raman spectral features of the measured Raman scattered elements.

27. The apparatus of claim 23 wherein the varying stimulus is one or more of: a non-optical or non-light stimulus; an ultrasound stimulus; a magnetic stimulus; a microwave stimulus; a time varying ultrasound field stimulus; a time varying magnetic field stimulus; a time varying light field stimulus; a time varying microwave or terahertz wave field stimulus; and an externally controlled time varying sample temperature stimulus.

28. The apparatus of claim 23 wherein the stimulation source comprises a high-intensity focussed ultrasound (HIFU) transducer having a central aperture, and one or both of the entry region and collection region are located within or visible through the central aperture.

29. The apparatus of claim 23 further comprising one or both of said SERS probes and said diffusely scattering sample.

30. A computer readable medium comprising computer program code for carrying out the following steps when executed on a suitable computer system:
receiving a timing signal representing a time varying stimulus which is provided to surface enhanced Raman Spectroscopy (SERS) probes within a sub-surface volume of a diffusely scattering sample, the SERS probes being spectrally responsive to the stimulus;
receiving data representing measured Raman scattered elements of probe light collected from said SERS probes during application of the time varying stimulus;
detecting from the timing signal and the data representing measured Raman scattered elements, variations in the measured Raman scattered elements which are induced by the variations in the stimulus; and
detecting one or more properties of the sample based on the detected variations in the measured Raman scattered elements.

31. The computer readable medium of claim 30 wherein detecting the variations in the measured Raman scattered elements which are induced by variations in the stimulus comprises detecting variations that are in synchrony with, or are correlated with, the timing signal.

32. The computer readable medium of claim 30 wherein the variations in the measured Raman scattered elements induced by variations in the stimulus comprise variations in one or more of the following aspects of one or more Raman spectral features of the measured Raman scattered elements: the spectral shape or form, the relative intensity, the width, or the wavenumber.
